(19) United States
(12) Patent Application Publication
(10) Pub. No.: US 2006/0084672 A1
(43) Pub. Date: Apr. 20, 2006

(54) Method for modification of y-secretase activity through inhibition of Fkbp13

(75) Inventor: Gabrielle Boulianne, Toronto (CA)

Correspondence Address: MCHALE & SLAVIN, P.A., 2855 PGA BLVD, PALM BEACH GARDENS, FL, 33410, US

(73) Assignee: The Hospital for Sick Children, Toronto (CA)

(21) Appl. No.: 10/970,054

(22) Filed: Oct. 20, 2004

(51) Int. Cl. A61K 31/4745

(52) U.S. Cl. 514/291

(57) ABSTRACT

The present invention provides a method for modifying γ-secretase activity in a cell or in an organism by de-stabilizing presenilin through inhibition of Fkbp13. Inhibition of Fkbp13 de-stabilizes presenilin and prevents formation of a functional γ-secretase complex thus decreasing γ-secretase activity and concomitantly decreasing production of amyloid-β peptides. The invention also provides methods for identifying agents which function to inhibit Fkbp13.

### FIELD OF THE INVENTION

The instant invention relates generally to protein-protein interactions that regulate proteolytic activity; particularly to methods for modification of protein-protein interactions to achieve control of the regulation of proteolytic activity; and most particularly to a method for modifying γ-secretase activity in a cell or in an organism by de-stabilizing presenilin through inhibition of Fkbp13 and thus preventing the formation of a functional γ-secretase complex. Amyloid Precursor Protein (APP) is unable to undergo proteolytic processing without the presence of a functioning γ-secretase complex, thus with inhibition of the γ-secretase complex, the build up of amyloid β-peptides in a cell or in an organism is concomitantly reduced.

### BACKGROUND OF THE INVENTION

Alzheimer's Disease (AD) is a common brain disorder that is becoming increasingly prevalent as the number of persons in the ageing population increases. The hallmark of AD is a gradual loss of cognitive functions resulting from the progressive degeneration and death of neurons in the brain. Symptoms of AD include memory impairment, loss of language, loss of reasoning ability, behavior changes and dementia. Histological identification of neurofibrillary tangles in degenerated neurons and/or extracellular neural plaques represent physical evidence of AD. The neural plaques consist of β-amyloid peptides which accumulate as a result of abnormal processing of β-amyloid precursor protein (APP) by the enzymatic action of the multi-protein γ-secretase complex. Reducing the deposition of β-amyloid peptides is generally believed to be key to alleviating the symptoms of and/or preventing the development of AD.

Presenilin protein is a particularly relevant component of the multi-protein γ-secretase complex. Presenilin proteins are highly conserved proteins that were first identified as causative factors in the pathology of Alzheimer's Disease and have since been shown to play a critical role in development by regulating the Notch signaling pathway (Selkoe et al. Annual Review of Neuroscience 26:565-597 2003). Presenilins are synthesized as precursor proteins that are rapidly cleaved by an unidentified “presenilase” to generate both an amino and a carboxy terminal fragment, each of which is required for function (Thinakaran et al. Neuron 17:181-190 1996). The large precursor is highly unstable and must be incorporated into a larger complex for stabilization (Ratovitski et al. Journal of Biological Chemistry 272:24536-24541 1997; Thinakaran et al. Journal of Biological Chemistry 272:28415-28422 1997; Capell et al. Journal of Biological Chemistry 273:3205-3211 1998; Tomita et al. Journal of Biological Chemistry 276:33273-33281 2001). The exact molecular weight and composition of this complex is unknown, although recent studies have implicated Nicastrin (Yu et al. Nature 407:48-54 2000; Hu et al. Dev. Cell 2:69-78 2002; Edbauer et al. PNAS USA 99:8666-8671 2002) and APH-1 (Goutte et al. PNAS USA 99:775-779 2002; Francis et al. Dev. Cell 3:923-935 2002; Lee et al. Journal of Biological Chemistry 277:45013-45019 2002; Gu et al. Journal of Biological Chemistry 278:7374-7380 2003) as factors required to stabilize presenilin fragments (Edbauer et al. PNAS USA 99:8666-8671 2002; Takasugi et al. Nature 422:438-441 2003). In addition to these two factors, presenilins also bind PEN-2 (Francis et al. Dev. Cell 3:923-935 2002; Steiner et al. Journal of Biological Chemistry 277:39062-39065 2002) and together all four proteins are thought to function as γ-secretase, a protease that can cleave type I transmembrane proteins including Notch and APP (De Strooper et al. Nature 398:518-522 1999; De Strooper et al. Nature 391:387-390 1998). In the case of Notch, cleavage within the transmembrane domain releases an intracellular domain, which can translocate to the nucleus, associate with transcription factors and regulate gene transcription. Consistent with this model, recent studies have shown that the expression of Presenilin, APH-1, PEN-2 and Nicastrin (Nct) in yeast can reconstitute γ-secretase activity (Edbauer et al. Nature Cell Biology 5:486-488 2003). The instant inventors hypothesized that the inhibition of an individual protein of the γ-secretase complex may prevent the formation of a functional enzyme and thus reduce production of β-amyloid peptides.

If a method can be devised for regulation of the γ-secretase activity by preventing the formation of a functional complex, it may also be possible to regulate the production of β-amyloid peptides, thus representing a novel avenue for the development of new compounds for the prevention and/or treatment of AD.

### DESCRIPTION OF THE PRIOR ART

Reducing the deposition of β-amyloid peptides is generally believed to be key to alleviating the symptoms of and/or preventing the development of AD. Since the γ-secretase is critical to the production and deposition of these β-amyloid peptides, it has been intensely studied, thus research has generated many methods for modification of γ-secretase, several examples of which are noted below.

U.S. Pat. No. 6,713,248 B2 (Roberts et al.) discloses a method for identification of γ-secretase inhibitors wherein a test compound is introduced into a sample containing uncleaved βAPP, βAPP fragments and γ-secretase. The γ-secretase is activated and the effect of the test compound on the amount of γ-cleaved βAPP fragments produced is monitored.

US Patent Application Publication 00228673 A1 2004 (Netzer et al.) discloses a method for inhibition of γ-secretase by inhibiting ATP (adenosine triphosphate) as γ-secretase activity requires ATP (to produce β amyloid peptides).

US Patent Application Publication 00224203 A1 2004 (Churcher et al.) discloses benzodiazepine derivatives which inhibit γ-secretase by modulating the processing of APP by γ-secretase. Methods for use of these compounds in the prevention and treatment of AD are also disclosed.

Weggen et al. (Journal of Biological Chemistry 278(34):31831-31837 2003) disclose that nonsteroidal anti-inflammatory drugs (NSAIDS) decrease β-amyloid production by modulating γ-secretase activity.

Netzer et al. (PNAS USA 100(21):12444-12449 2003) disclose that the Ab1 kinase inhibitor imatinib mesylate (GLEEVEC or ST1571), which targets the ATP-binding site of Ab1 and other tryosine kinases, reduces the production of β-amyloid since γ-secretase activity is ATP-dependent.

Petit et al. (Journal of Neuroscience Research 73(3):370-377 2003) disclose that JKL isocoumarin inhibitors function as γ-secretase inhibitors that can selectively reduce the production of β-amyloid without affecting other proteins.

Wolfe et al. (Journal of Molecular Neuroscience 19(1-2):83-87 2002) disclose hydroxyethyl urea peptidomimetics that inhibit γ-secretase activity by mimicking the APP substrate conformation.

Generally, methods and compositions for achieving a reduction in β-amyloid peptides are aimed at control of the cleavage of APP by γ-secretase or inhibition of the ATP energy requirements of γ-secretase necessary for cleavage of APP. The method of the instant invention is designed to achieve inhibition of γ-secretase by preventing the formation of an active γ-secretase complex by de-stabilizing presenilin. The instant inventors are the first to carry out inhibition of Fkbp13 to de-stabilize presenilin thus preventing formation of a functional γ-secretase complex and concomitantly decreasing production of β-amyloid peptides in a cell.

### SUMMARY OF THE INVENTION

The three-dimensional molecular conformation of a protein is essential to its proper functioning. Molecular shape allows for the recognition of a protein by other proteins which is necessary for protein-protein interaction. Conformation also provides a means for regulation of the accessibility of reactive amino acid residues. A protein must maintain a stable molecular conformation for interaction or reaction to occur. Frequently, proteins associate with other proteins to achieve the stabilization of structure required to assure proper functioning.

The abnormal processing of APP by γ-secretase results in the deposition of the β-amyloid peptides which have been identified in AD. Four proteins (presenilin, APH-1, PEN-2 and nicastrin) associate to function as γ-secretase. Inhibition of the γ-secretase activity can decrease the production of β-amyloid peptides and thus potentially prevent and/or ameliorate the symptoms of AD.

The instant inventors were the first to discover that Fkbp13 protein stabilizes presenilin protein in the endoplasmic reticulum allowing for formation of a functional γ-secretase complex and that inhibition of the interaction between Fkbp13 protein and presenilin protein prevents the formation of a functional γ-secretase complex, thus decreasing γ-secretase activity and concomitantly decreasing production of amyloid-β peptides. Modification of γ-secretase activity through inhibition of Fkbp13 protein provides a novel approach for the prevention and/or treatment of AD and provides an avenue for the development of new compounds for use in prevention and therapy of AD.

Accordingly, it is an objective of the instant invention to provide a method for modifying Fkbp13 protein interaction with presenilin protein in a cell or an organism by providing a composition including at least one Fkbp13 protein inhibitor and administering the composition to the cell or to the organism in an amount effective to achieve modification of the Fkbp13 protein interaction with presenilin protein in the cell or in the organism wherein said modification produces a decrease in γ-secretase activity.

It is another objective of the instant invention to provide a method for modifying Fkbp13 protein interaction with presenilin protein in a cell or an organism by providing a composition including FK506 and administering the composition to the cell or to the organism in an amount effective to achieve modification of the Fkbp13 protein interaction with presenilin protein in the cell or in the organism wherein said modification produces a decrease in γ-secretase activity.

It is yet another objective of the instant invention to provide a method for modifying Fkbp13 protein interaction with presenilin protein in a cell or in an organism by providing a composition including rapamycin and administering the composition to the cell or to the organism in an amount effective to achieve modification of the Fkbp13 protein interaction with presenilin protein in the cell or in the organism wherein said modification produces a decrease in γ-secretase activity.

It is another objective of the instant invention to provide a method for de-stabilizing presenilin protein in order to prevent formation of a functional γ-secretase complex and to reduce production of β-amyloid peptides.

It is still another objective of the instant invention to provide a pharmaceutical composition for modifying Fkbp13 protein interaction with presenilin protein in a cell or in an organism comprising at least one Fkbp13 protein inhibitor combined with a pharmaceutically acceptable solution.

It is still another objective of the instant invention to provide a pharmaceutical composition for modifying Fkbp13 protein interaction with presenilin protein in a cell or in an organism comprising FK506 combined with a pharmaceutically acceptable solution.

It is still another objective of the instant invention to provide a pharmaceutical composition for modifying Fkbp13 protein interaction with presenilin protein in a cell or in an organism comprising rapamycin combined with a pharmaceutically acceptable solution.

It is yet another objective of the instant invention to provide a method for reducing β-amyloid production in a cell or in an organism by providing a composition including at least one Fkbp13 protein inhibitor wherein said at least one Fkbp13 protein inhibitor modifies an interaction between Fkbp13 protein and presenilin protein and administering the composition to the cell or to the organism in an amount effective to achieve modification of said interaction between said Fkbp13 protein and said presenilin protein wherein said modification produces a decrease in γ-secretase activity with a concomitant reduction in production of β-amyloid peptides.

It is yet another objective of the instant invention to provide a method for reducing β-amyloid production in a cell or in an organism by providing a composition including FK506 wherein said FK506 modifies an interaction between Fkbp13 protein and presenilin protein and administering the composition to the cell or to the organism in an amount effective to achieve modification of said interaction between said Fkbp13 protein and said presenilin protein wherein said modification produces a decrease in γ-secretase activity with a concomitant reduction in production of β-amyloid peptides.

It is still another objective of the instant invention to provide a method for reducing β-amyloid production in a cell or in an organism by providing a composition including rapamycin wherein said rapamycin modifies an interaction between Fkbp13 protein and presenilin protein and administering the composition to the cell or to the organism in an amount effective to achieve modification of said interaction between said Fkbp13 protein and said presenilin protein wherein said modification produces a decrease in γ-secretase activity with a concomitant reduction in production of β-amyloid peptides.

It is still another objective of the instant invention to provide a method for identifying an agent that inhibits an interaction between Fkbp13 protein and presenilin protein in a cell by administering an agent to be assessed for its ability to inhibit an interaction between Fkbp13 protein and presenilin protein in a cell; determining the extent to which inhibition of an interaction between Fkbp13 protein and presenilin protein occurs in the cell to which the agent is administered and comparing the extent of inhibition determined to the extent of inhibition in an appropriate control cell wherein if said inhibition occurs to a greater extent in the cell to which the agent is administered than in the control cell, the agent inhibits an interaction between Fkbp13 protein and presenilin protein in the cell. It is also an objective of the instant invention to provide agents identified by this method. As used herein, the term “agent” refers to any substance which can be assessed for modification of protein-protein interactions. In the instant case, the “agent” may be a peptide, a drug or any other substance capable of inhibiting an interaction between Fkbp13 and presenilin.

Other objectives and advantages of the instant invention will become apparent from the following description taken in conjunction with the accompanying drawings wherein are set forth, by way of illustration and example, certain embodiments of the instant invention. The drawings constitute a part of this specification and include exemplary embodiments of the present invention and illustrate various objects and features thereof.

### ABBREVIATIONS AND DEFINITIONS

The following list defines terms, phrases and abbreviations used throughout the instant specification. Although the terms, phrases and abbreviations are listed in the singular tense the definitions are intended to encompass all grammatical forms.

As used herein, the term “modification” refers to any action and/or treatment which alters the function of a protein.

As used herein, the term “inhibition” refers to any action and/or treatment which operates against the full activity of a protein thus reducing and/or completely suppressing protein function.

As used herein, the term “interaction” refers to an action wherein two substances in close physical proximity act upon each other.

As used herein, the term “de-stabilization” refers to any action and/or treatment which affects the structure and/or conformation of a protein thus preventing its ability to engage in normal interactions.

As used herein, the phrase “amount effective” refers to an amount of a composition sufficient to elicit a change in activity of the γ-secretase complex.

As used herein, the abbreviation “AD” refers to Alzheimer's Disease, a common brain disorder characterized by a gradual loss of cognitive functions resulting from the progressive degeneration and death of neurons in the brain.

As used herein, the term “Fkbp13” refers to an enzyme, named for its calculated molecular mass, which binds the immunosuppressant drug, FK506. Fkbp13 is a peptidyl-prolyl isomerase which functions to catalyze isomerization between the cis and trans forms of the Xaa-Pro peptide bond. It is not known if this isomerase function is important in stabilizing presenilin. Fkbp13 is localized to the endoplasmic reticulum where it functions in folding polypeptide chains as they are synthesized.

As used herein, the abbreviation “PPIase” refers to a peptidyl-prolyl isomerase.

As used herein, the term “immunophilin” refers to a family of proteins whose function is blocked by binding with immunosuppressant drugs.

As used herein, the abbreviation “ER” refers to the endoplasmic reticulum, a membrane system located within eukaryotic cells which functions in the synthesis of biomolecules.

As used herein, the term “golgi apparatus” refers to a membrane system located within eukaryotic cells which functions in processing and secretion of biomolecules.

As used herein, the term “FK506” refers to a metabolic product of a soil fungus (Streptomyces Tsukabaensis) with potent immunosuppressant activity. FK506 inhibits T lymphocyte activation by inhibiting calcium/calmodulin dependent serine/threonine phosphatases thus blocking signal transduction which leads to the production of cytokines necessary for perpetuation and amplification of an immune response.

As used herein, the term “γ-secretase” refers to a multi-protein enzyme which catalyzes cleavage of type I transmembrane proteins, such as Notch and Amyloid Precursor protein. Nicastrin (Nct), presenilin, PEN-2 and APH-1 are required for γ-secretase function.

As used herein, the term “presenilin” refers to an integral membrane protein predominantly localized within the ER and the Golgi of neurons within the central nervous system (CNS). Presenilin associates with the proteins APH-1, Nicastrin and PEN-2 to constitute the γ-secretase activity which cleaves APP to produce 42 residue β-amyloid peptide, a major component of the plaques of AD. There are two forms of presenilin, designated presenilin-1 (PS1) and presenilin-2 (PS2). Mutations in either presenilin gene (PSEN1 or PSEN2) lead to a rapid increase in β-amyloid peptide deposition. The term “presenilin” and the abbreviation “PSN” are used herein to refer to either PS1, PS2 or to both PS1 and PS2.

As used herein, the abbreviation “APP” refers to Amyloid Precursor Protein, a transmembrane protein that is abnormally processed by γ-secretase to release the β-amyloid peptides which are central to the pathogenesis of AD. Deposition of β-amyloid peptides form the neural plaques found in AD.

As used herein, the term “Notch” refers to a transmembrane protein that is involved in signaling pathways which regulate gene transcription and cell-cell signaling. Notch is particularly important during development.

As used herein, the term “ryanodine receptor” encoded by the RyR gene, refers to a calcium release channel found in the ER. The Fkbp13 gene encodes an isomerase that binds to and regulates the gating of the ryanodine receptor.

As used herein, the term “agent” refers to any substance which can be assessed for modification of protein-protein interactions. In the instant case, the “agent” may be a peptide, a drug or any other substance capable of inhibiting an interaction between Fkbp13 and presenilin.

The phrase “pharmaceutically acceptable” is used herein as described in U.S. Pat. No. 6,703,489. “Pharmaceutically acceptable” means approved by a regulatory agency or listed in a generally approved pharmacopeia for use in animals and humans. Solutions are usually preferred when a composition is administered intravenously. Illustrative, albeit non-limiting examples of pharmaceutically acceptable solutions include water, oils, saline, aqueous dextrose and glycerol.

As used herein, the term “isolated peptide” refers to a peptide which has been “altered by the hand of man” and separated from the co-existing materials of its natural state. An isolated peptide has been changed or removed from its original environment or both.

The terms “β-amyloid peptide” and “amyloid-β peptide” are used interchangeably herein.

### BRIEF DESCRIPTION OF THE FIGURES

The instant patent or application file contains at least one drawing executed in color. Copies of this patent or patent application publication with color drawing(s) will be provided by the Office upon request and payment of the necessary fee.

FIGS. 1A-H show micrographs of Drosophila phenotypes due to misexpression of presenilin and the effect of loss-of-function mutations in RyR and Fkbp13 on these phenotypes.

FIGS. 2A-C show results of experiments indicating that presenilin associates with Fkbp13 and Rya-r44f.

FIGS. 3A-F show results of experiments indicating that Psn protein but not RNA levels are reduced in Fkbp13 mutants.

FIGS. 4A-B show results of experiments indicating that FK506 reduces both PEN2 protein levels and amyloid-β peptide production.

FIGS. 5A-F show micrographs illustrating that Fkbp13 is an ER-resident protein.

FIG. 6 show results of coimmunoprecipitation experiments indicating that GFP-Rya can pull down both Psn and Fkbp13.

### DETAILED DESCRIPTION OF THE INVENTION

### EXAMPLE 1

Loss-of-Function Mutations in RyR and Fkbp13 Suppress Phenotypes Due to Misexpression of Presenilin

In order to gain further insight into the function of Presenilin and to identify other components of the Presenilin pathway, the instant inventors first searched for Presenilin-interacting genes in Drosophila. Specifically, the instant inventors sought to identify enhancers and suppressors of two dominant adult phenotypes generated by misexpression (overexpression) of wildtype Drosophila presenilin (psn)in third larval instar wing imaginal discs.

Recombinant transgenic lines expressing a UAS::psn transgene and either a Cut:GAL4 (wing) or Pnr::GAL4 (notum) driver were generated and used to screen a collection of lethal mutations generated by P or EP-element insertion. The line EP(2)2019 and EP(2)2206 represent insertions of an EP-element in the first intron or in the 5′ upstream region of Fkbp13, respectively. EP(2)2019 was used to generate a null mutation in Fkbp13 (Fkbp13^{D58}) by imprecise excision. Fkbp13^{D34} and Rya-r44f^{H3} represent precise excisions for Fkbp13 and Rya-r44f, respectively. Psn^{w6rP} is a null mutation in psn (Guo et al. Journal of Neuroscience 19:8435-8442 1999). Oregon-R was used as a wildtype control.

The open reading frames (ORFs) of Fkbp13 and psn were amplified from EST clones obtained from BDGP (Berkeley Drosophila Genome Project) using pfuTurbo DNA polymerase (Stratagene). A full-length GFP-tagged Rya-r44f cDNA was obtained from Xu et al. (Biophysical Journal 78:1270-1281 2000). The following constructs were then generated in either pcDNA3 or pcDNA3.1 (Invitrogen):pcDNA-GFP-Rya-r44f, pcDNA3.1-Xpress-Fkbp13 and pcDNA3.1-HA-psn.

HEK293 cells were maintained at 37° C. and 5% CO₂ in α-minimal essential medium (Hyclone) supplemented with 10% heat-inactivated fetal bovine serum and plated 24 hours before transfection. For each 10 cm tissue culture dish, 12-16 μg cDNA was used. HEK293 cells were transiently transfected with plasmids encoding Drosophila Rya-r44f, Fkbp13, or psn using calcium phosphate precipitation. The cells were harvested 40-42 hours afer transfection.

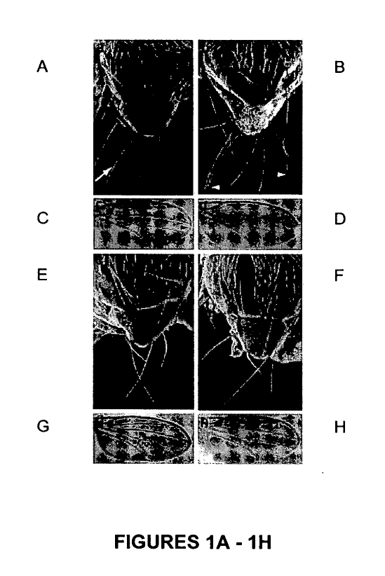

Transgenic lines were generated in which a full-length psn transgene was expressed under the transcriptional control of the GAL4-UAS system (Brand et al. Development 118:401-415 1993). Adult phenotypes were generated in the scutellum and wing using two independent GAL4 lines to drive expression of a UAS-psn transgene (FIGS. 1A-H). Wildtype adult flies normally have four large bristles, termed macrochaetes on their scutellum (arrow points out bristles in FIG. 1A) and a smooth wing margin (black arrowhead points out smooth wing margin in FIG. 1C). Using a pnr-GAL4 line that drives expression in the thoracic region of the wing imaginal disc (Ramain et al. Development 119:1277-1291 1993), or a cut-GAL4 line to drive expression of UAS-psn along the presumptive wing margin, it was found that overexpression of psn results in supernumerary macrochaetes on the scutellum (white arrows point out supernumerary bristles in FIG. 1B) and a notched wing phenotype similar to that observed in Notch loss-of-function mutants (black arrowhead points out the notched wing in FIG. 1D). The observation that overexpression of psn resembles the loss-of-function psn phenotype has previously been described and attributed to possible dominant-negative effects (Ye et al. Journal of Cell Biology 146:1351-1364 1999). While the precise mechanisms leading to this phenotype are presently unknown, it has been proposed that it may result from the accumulation of non-functional complexes within the ER/Golgi compartments that disrupt endogenous Psn function. Both phenotypes are 100% penetrant at 29° C. and were used to screen a collection of P-element lethal insertions for those that could enhance or suppress both phenotypes. It was found that loss-of-function mutations caused P-element insertions in the genes encoding the Drosophila Ryanodine Receptor, Rya-r44f (1(2)k04913) and Fkbp13 (EP(2)2019 and EP(2)2206), suppress the psn phenotype in the notum and wing of adult flies. FIGS. 1E and 1G show suppression of psn phenotypes by EP(2)2019, a hypomorphic mutation in Fkbp13. FIGS. 1F and 1H show suppression of psn phenotypes by Rya-r44f¹⁰⁴⁹¹3, a loss-of-function mutation in Ryanodine receptor 44F. The RyR gene encodes a calcium release channel found primarily in the endoplasmic reticulum (ER) whereas, Fkbp13, encodes a proline isomerase that has been shown to bind to and regulate normal gating of the RyR (Brillantes et al. Cell 77:513-523 1994). In order to confirm that the P-element insertion mutations in Rya-r44f and Fkbp13, rather than second site mutations, were responsible for suppressing the psn phenotypes, precise excisions for two of the P-element lines were generated. In both cases, the precise excision lines no longer suppressed the psn phenotypes demonstrating that mutations in the Rya-r44f gene and Fkbp13 are responsible for the genetic suppression of psn (this data is not shown).

### EXAMPLE 2

Genetic Interactions Between Presenilin and Rya-r44f and/or Fkbp13

In order to determine if genetic interactions between psn and Rya-r44f or Fkbp13 are a result of protein-protein interactions, co-immunoprecipitation experiments were performed.

Protein quantification, SDS-polyacrylamide gel electrophoresis (5-12% or 8-12% gradient gel, or 10% SDS-PAGE) and immunoblot analysis were carried out as described previously (Guo et al. Journal of Cell Science 113:3499-3508 2000) using the monoclonal antibodies, anti-β-actin and anti-FLAG (Sigma), anti-GFP (Santa Cruz Biotechnology), anti-HA (12c-A5, Roche), anti-β-tubulin (Developmental Studies Hybridoma Bank), anti-Xpress and anti-V5 (Invitrogen) and a polyclonal anti-GFP (Santa Cruz Biotechnology). Microsomal fractions were prepared from cells using a detergent-free protocol. All steps were performed at <4° C. Cell pellets were resuspended in 300 μl hypo-osmotic buffer (20 mM Tris/HCl, pH 7.5) supplemented with complete protease inhibitor (Roche), homogenized through a 27G needle and sonicated. Nuclei were removed by centrifugation (1200 g, 25 minutes) and the post-nuclear supernatant (containing membrane and soluble fractions) was denatured for 5 minutes at 100° C. in SDS-PAGE sample buffer. For co-immunoprecipitation experiments, microsomal fractions were obtained from the post-nuclear supernatant by centrifugation at 100,000 g for 1 hour at 4° C. and the pellets were resuspended in buffer (50 mM Tris/HCl, pH 7.5).

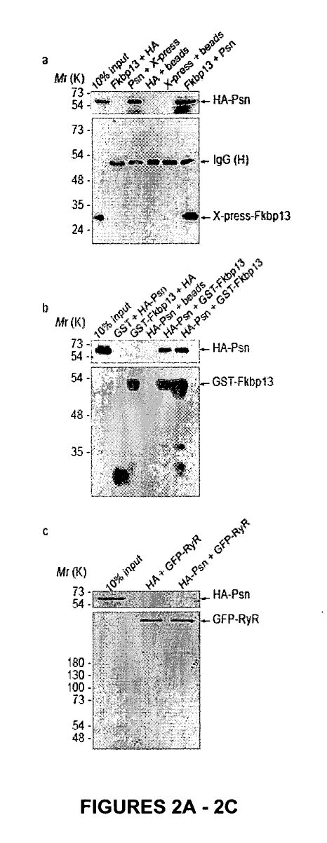

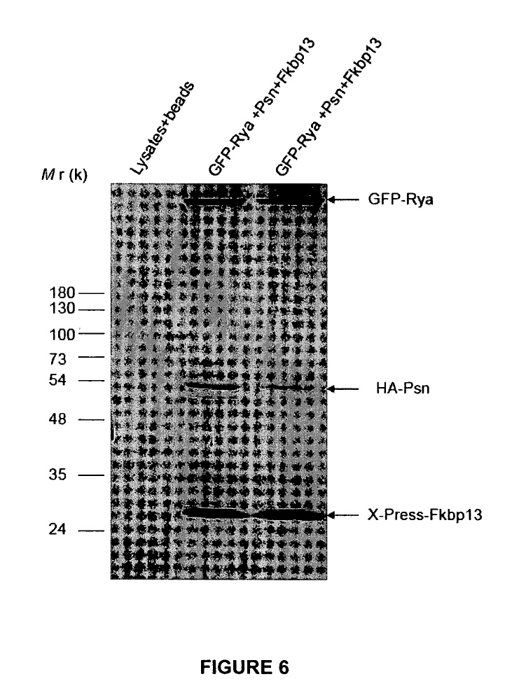

Tagged versions of psn, Rya-r44f and Fkbp13 were co-transfected into HEK293 cells. It was found that Rya-r44f and Fkbp13 co-immunoprecipitate as do Rya-r44f and psn (FIG. 6) consistent with previous findings in vertebrates (Brillantes et al. Cell 77:513-523 1994; Chan et al. Journal of Biological Chemistry 275:18195-18200 2000). It was also found that psn co-immunoprecipitates with Fkbp13 (FIG. 2A) demonstrating that these proteins can be found within a complex. The data shown in FIG. 2A resulted from an experiment wherein HEK cells were transfected with HA-tagged psn and X-press-tagged Fkbp13. Cell extracts were then prepared and immunoprecipitated with anti-HA antibodies (to detect psn) and then analyzed by immunoblotting with an anti-X-press antibody (to detect Fkbp13).

### EXAMPLE 3

Fkbp13 Directly Binds Presenilin

In order to determine if Psn binds directly to either Rya-r44f or Fkbp13, GST pull-down and affinity chromatography using purified proteins were carried out.

A Drosophila Fkbp13 cDNA corresponding to amino acids 298-828 was subcloned into the bacterial expression vector pGEX-4T-1/His 6C (Novagen) to produce Drosophila Fkbp13 GST-fusion protein and to raise polyclonal Fkbp13 antibodies. Antibody specificity was demonstrated by blocking with excess fusion protein and by showing that the antigen was depleted in Fkbp13 null mutants. GFP-Rya-r44f and HA-Psn were purified using a competitive immunoprecipitation (IP) procedure. Briefly, microsomal fractions were resuspended in IP binding buffer. 600 μg of the microsomal fraction was mixed with 1 μg of anti-GFP or anti-HA and incubated with protein A agarose (Invitrogen) for 1 hour at 4° C. The IP complexes were then washed with IP washing buffer. The GFP-Rya-r44f or HA-Psn were eluted by adding 5× excess of in vitro translated GFP or HA protein (TNT Coupled Reticulocyte lysate Kit; Promega, Madison, Wisc., USA) and rotated for 2 hours at 4° C. The supernatants were then used in the GST pull-down assay as described (Guo et al. Journal of Cell Science 113:3499-3508 2000).

Purified Psn directly bound to immobilized Fkbp13 (FIG. 2B) but not to the Rya-r44f (FIG. 2C). FIG. 2B shows GST-pull downs that were used to demonstrate that Psn binds directly to Fkbp13. Cell extracts were prepared from cells expressing HA-tagged Psn and incubated with beads containing GST-HA-Psn, GST-Fkbp13 or GST alone. Psn bound specifically to GST-Fkbp13 but not to the negative controls. FIG. 2C shows results from the affinity chromatography which was used to determine the ability of Psn to bind Rya-r44f. Both Rya-r44f and Psn can both be co-immunoprecipitated from these extracts, however in vitro translated HA-Psn does not bind to purified GST-Rya-r44f. Consistent with previous studies, it was also determined that Fkbp13 can bind directly to the Rya-r44f (this data is not shown). The results of these experiments suggest that Fkbp13 directly binds Psn and may mediate the interaction between Psn and the Rya-r44f, thus it also may be possible to manipulate RyR to effect psn.

### EXAMPLE 4

Molecular Basis of the Interaction Between Presenilin and Fkbp13

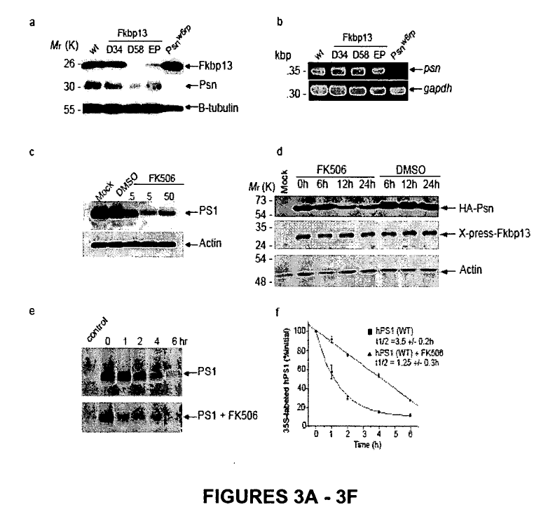

In order to further characterize the molecular basis of the interaction between Psn and Fkbp13, null mutations in Fkbp13 were generated by imprecise excision of the EP-element EP(2)2019. Specifically, a 2.4 kb deletion, Fkbp13^{D58}, which removes the first and second exons of Fkbp13 including the transcriptional start site, was generated. To confirm that Fkbp13^{D58} represents a null mutation in Fkbp13, an antibody to an Fkbp13 fusion protein was generated. Fkbp13 levels are severely reduced in the original EP-element insertion line, EP(2)2019 and absent from the deletion mutant Fkbp13^{D58} confirming that this represents a null mutation in Fkbp13 (FIG. 3A). FIG. 3A shows immunoblots of extracts prepared from wildtype and mutant pupae. Fkbp13 levels are absent from the null mutant Fkbp13^{D58} and severely reduced in the hypomorphic allele, Fkbp13^{EP(2)2019} as compared to wildtype controls and the precise excision line, Fkbp13^{D34}. Fkbp13 levels area also normal in the psn null mutant, psn^{w6rp}. Similarly, Psn levels are also normal in wildtype and Fkbp13^{D34} but are reduced by up to 80% in both Fkbp13 mutants and absent in the psn null mutant. Anti-β-tubulin antibody was used as a loading reference. Immunoblots were performed in triplicate and protein levels quantified using a densitometer.

Both the original EP insertion in Fkbp13 and the null mutant Fkbp13^{D58} mutants are homozygous lethal and die as pharaete adults. Both the hypomorphic allele, EP(2)2019 and the null mutant Fkbp13^{d58} display defects in both eye and bristle development with a rough eye phenotype consisting of slightly disorganized ommatidia and missing interommatidial bristles. Similarly, a severe disruption of bristles on the notum of mutants was observed as compared to wildtype controls or the precise excision, Fkbp13^{D34} (this data is not shown). Such defects in eye and bristle development are indicative of defects in Notch signaling and could reflect a direct requirement for Fkbp13 in regulating Notch function. Alternatively, since it was found that Fkbp13 binds to Psn, loss-of-function mutations in Fkbp13 could be giving rise to defects in Notch signaling by affecting Psn function. In order to distinguish between these possibilities, Psn levels were examined in Fkbp13 mutants. Psn protein, but not RNA levels, were reduced by up to 80% in Fkbp13 null mutants (FIGS. 3A-B). The RT-PCR analysis of psn gene expression in FIG. 3B shows that psn transcripts are normal in the Fkbp13 mutants compared to controls but reduced in psn mutants. GAPDH was used as a loading control.

Fkbp13 levels are unaffected by null mutations in psn. These results demonstrate that Fkbp13 binds to, and affects Psn protein stability, thereby leading to defects in Notch signaling.

### EXAMPLE 5

Fkbp13 is Localized to the Endoplasmic Recticulum (ER)

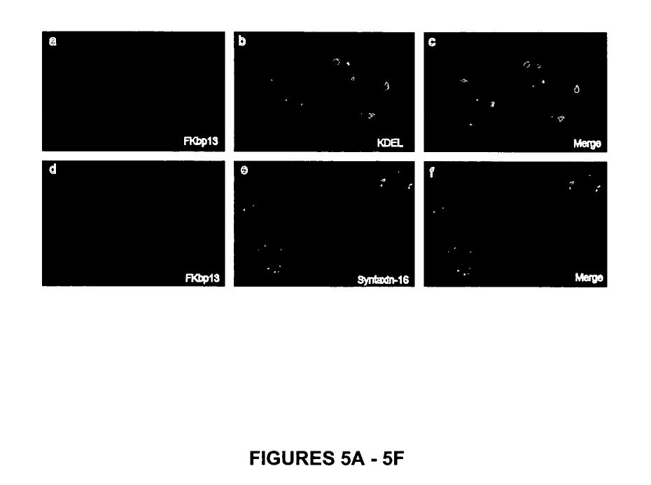

The antibody to an Fkbp13 fusion protein generated in Example 4 was used to demonstrate that, like its vertebrate counterpart, Drosophilia Fkbp13 was localized to the endoplasmic reticulum (FIGS. 5A-F). Drosophilia S2 cells were double-labelled with antibodies to Fkbp13 and either an ER marker (anti-KDEL, FIGS. 5A-C) or a Golgi marker (anti-syntaxin-16, FIGS. 5D-F). Fkbp13 only co-localized with the ER marker. Since Fkbp13 is an ER resident protein, Fkbp13 interaction with the γ-secretase complex may occur in the ER.

FIGS. 5A and 5D show cells stained with the anti-Fkbp13 antibody. FIG. 5B shows cells stained with the anti-KDEL antibody. FIG. 5C shows the results of a merge between cells stained with the anti-Fkbp13 antibody and the anti-KDEL antibody. FIG. 5E shows cells stained with the anti-syntaxin-16 antibody. FIG. 5F shows the results of a merge between cells stained with the anti-Fkbp13 antibody and the anti-syntaxin-16 antibody.

### EXAMPLE 6

FK506 Inhibits Binding of Fkbp13 to Presenilin, Destabilizing Presenilin and Functioning as a γ-Secretase Inhibitor

Fkbp13 belongs to a family of proteins termed immunophilins, which were identified by virtue of their binding to the drug, FK506. Previous studies have shown that FK506 can compete Fkbp12 off of the RyR. Thus, it may be possible that FK506 can inhibit binding of Fkbp13 to Psn, de-stabilize the protein (psn) and thereby function as a γ-secretase inhibitor. In order to test this assumption directly, the effect of FK506 on Psn levels(both transfected and endogeneous) was examined in HEK293 cells.

To determine the effect of drug treatment on Psn stability, HEK293 cells were transiently co-transfected with psn and Fkbp13 constructs. 24 hours after transfection, 100 mm dishes of transfected HEK293 cells were treated with 5 μM FK506 (CALBIOCHEM), 1 μM Rapamycin (CALBIOCHEM) or DMSO (control), harvested at various time points and analyzed by Western blotting techniques. To measure Aβ40, Cos-7 cells were transfected with pCEP4-SPA4CT-DA (gift from Dr. Gunter Merdes, ZMBH, University of Heidelberg) that contains an APP^{C99} cDNA using lipofectin (Gibco) according to the manufacturers protocol. Cells stably expressing APP^{C99} were selected using 300 μg/ml hygromycin and maintained in DMEM medium containing 10% FBS, lbopg/ml penicillin-streptomycin and 100 μg/ml hygromycin. These cells were then transiently transfected with plasmids carrying human PS1. 48 hours after transfection, cells were starved in serum-free DMEM medium for 2 hours, then treated with 1 μm of the γ-secretase inhibitor DAPT or 0.5 μm of the Fkbp13 inhibitor rapamycin or FK506 respectively, in DMEM medium containing 5% FBS o/n. Cells then were harvested and lysed and the conditioned lysates were used to measure Aβ1-40 by sandwich ELISA as described in (Yan et al. Nature 402:533-537 1999)using 6E10 (Senetek) as the capture antibody and the polyclonal Pan-β-Amyloid (Oncogene) for detection of Aβ40. The Aβ42 levels were below the limits of detection.

It was found that FK506 reduced Psn in a dosage and time dependent manner (FIG. 3C). FIG. 3C demonstrates that FK506 causes this dose-dependent reduction in endogenous PS1 protein levels. HEK293 cells were treated with 0.5, 5 and 50 μM FK506 or with DMSO as a control for 24 hours at 37° C. Actin was also tested.

A similar reduction of PS1 levels was observed in cells transfected with a PS1 construct (FIG. 3D). HEK293 cells transfected with Fkbp13 and Psn were treated with FK506 or DMSO for 1-24 hours. FK506 caused a dose-dependent decrease in the levels of Psn but had no effect on Fkbp13 or Actin. Similarly, cells treated with DMSO as a control showed no decrease in the levels of Psn.

In contrast, FK506 had no effect on either Actin or Fkbp13 itself (FIGS. 3C and 3D).

### EXAMPLE 7

FK506 Reduces Presenilin Stability

In order to confirm that FK506 was affecting the stability of PS1 a pulse-chase of ³⁵S-methionine was performed and the amount of PS1 remaining after various chase periods was measured by immunoprecipitation and autoradiography.

Pulse-chase labeling of cells was performed using TRAN ³⁵S-LABEL™ Metabolic Labeling Reagent (ICN). Cos-7 cells were transiently transfected with human PS-1. After 48 hours, cells were washed with PBS and then starved in the presence of methionine and cysteine free DMEM (Invitrogen) containing 10% dialyzed fetal bovine serum for 1 hour at 37° C. Cells were then incubated with [³⁵S] methionine (400 μCi/ml) in the presence or absence of FK506 for an additional 2 hours. The cells were then washed with 10 ml of PBS twice and chased with DMEM containing the indicated reagents for various times. Cells were then harvested and lysed in IP buffer and TCA precipitated to quantify the incorporation of ³⁵S-methionine. Equal amounts of microsomal fractions from radioactive cells were then incubated with 2 μg of anti-human PS1. Subsequently, 30p1 of protein A agarose beads (Invitrogen) were added to the IP reactions and rotated for 2 hours at 4° C. The beads were then washed 4 times with IP washing buffer, denatured in 2× SDS-PAGE sample buffer and run on 12% SDS-PAGE.

Consistent with previous studies (Ratovitski et al. Journal of Biological Chemistry 272:24536-24541 1997) PS1 levels were found to decline relatively rapidly with a half-life of 3.5±0.2 h. The addition of FK506 causes a more rapid decline in PS1 levels with half-life being only 1.25±0.3 h (FIGS. 3E and 3F).

### EXAMPLE 8

Fkbp13 Affects the Stability of Other Components of the γ-Secretase Complex

In order to determine if Fkbp13 can also affect the stability of other components of the γ-secretase complex, Drosophilia S2 cells were transfected with tagged constructs expressing APH-1, PEN-2 or Nct and then examined for the effects of FK506 on protein levels.

cDNA's encoding APH-1, PEN-2 and Nct were subcloned into pAc5.1/V5-HisA or pIB/V5-His TOPO and transiently transfected into Drosophilia S2 cells using cellfectin according to the manufacturer's protocol (Invitrogen). Cells were collected between 24-48 hours for Western analysis using the following antibodies: monoclonal anti-V5 1:5000(Invitrogen), monoclonal anti-HA 1:2000(Roche), monoclonal anti-FLAG M2 1:5000(Sigma) and monoclonal anti-β-tubulin 1:1000 (DSHB).

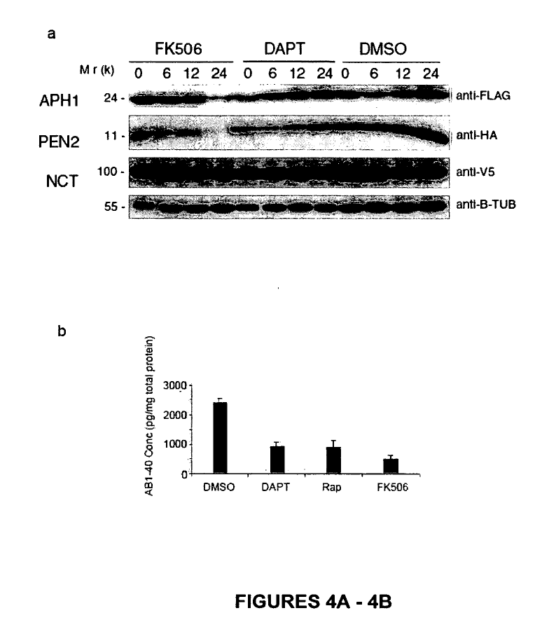

Similar to what was observed with PS1, Fk506 dramatically reduced the levels of PEN-2 (FIG. 4A). In contrast, no effect was observed on the levels of either APH-1 or Nct (FIG. 4A). FIG. 4A shows that FK506 causes a dose-dependent decrease in the levels of PEN-2. Drosophilia S2 cells were transfected with tagged versions of APH-1, PEN-2 or Nct and treated with 50 μM FK506 over a 24 hour period. No effect was observed on the levels of APH-1, Nct or β-tubulin control. Similarly, no effect was observed using either DMSO (control) or the known γ-secretase inhibitor, DAPT.

These results are also consistent with recent studies that showed PEN-2 is unstable in the absence of PS1 (Crystal et al. Biochemistry 43:3555-3563 2004; Bergman et al. Journal of Biological Chemistry 279:16744-16753 2004).

### EXAMPLE 9

FK506 is a γ-Secretase Inhibitor In Vivo

Since FK506 can reduce the levels of both PS1 and PEN-2, the instant inventors reasoned that FK506 would function as a γ-secretase inhibitor in vivo. In order to test this reasoning, FK506 was added to cells expressing both PS1 and APP^{C99} (a C-terminal fragment of human APP that is the target of γ-secretase activity).

Cos-7 cells stably expressing APP^{C99} were transiently transfected with human PS1 cDNA. The cells were then treated with DAPT (a known γ-secretase inhibitor) or the Fkbp13 inhibitors, rapamycin and FK506. Data was collected from two independent experiments, each in duplicate, and normalized to total proteins added. Error bars in FIG. 4B represent standard errors.

FK506 resulted in an 80% decrease in the levels of β-amyloid peptide as compared to untreated or DMSO treated controls (FIG. 4B). This is comparable to that observed using DAPT, a known γ-secretase inhibitor (FIG. 4B). Rapamycin was also observed to produce a decrease in β-amyloid peptide production (FIG. 4B). Rapamycin has previously been shown to inhibit the function of FK506-binding proteins. This data further demonstrates that FK506 can function as a γ-secretase inhibitor in vivo.

### CONCLUSIONS

Taken together, all of the data herein demonstrates that Fkbp13 binds to Psn and is required to maintain its stability. Removing Fkbp13 function either by creating null mutations or by adding the drug FK506, results in suppression of the psn overexpression phenotype due to a severe reduction of Psn protein and a concomitant loss of γ-secretase activity. The instant inventors hypothesize that a complex between Fkbp13 and Psn is necessary to stabilize Psn levels within the ER allowing for formation of the γ-secretase complex. This is consistent with previous studies demonstrating that Psn is highly unstable and must be incorporated into a larger complex for stabilization. Recent studies using a combination of misexpression and RNA interference techniques in human and Drosophila cells have shown that two of the components of the γ-secretase complex, APH-1 and Nicastrin, are also required for Psn stability (Takasugi et al. Nature 422:438-441 2003). In particular, these studies have shown that misexpression of APH-1 increase the stability of the holoprotein whereas depletion results in a loss of both Psn holoprotein and proteolytically cleaved fragments.

FK506-binding proteins have previously been shown to bind and stabilize the RyR thereby affecting gating of this ER calcium release channel (Ondrias et al. Annals of the NY Academy of Science 853:149-156 1998). Furthermore, studies have also shown that vertebrate Presenilin can co-immunoprecipitate with RyR3 and that cells that express FAD-linked mutations in Presenilins exhibit increased levels of RyR and enhanced calcium release that has been linked to an increase in neuronal cell death (Chan et al. Journal of Biological Chemistry 275:18195-18200 2000). Consistent with these results, the instant inventors found that Drosophila Psn can also co-immunoprecipitate with both Fkbp13 and the Rya-r44f. However, only Fkbp13 appears to bind directly to Psn. The instant inventors therefore hypothesize that the interaction between the Rya-r44f and Psn is mediated through Fkbp13.

It is well-established that the abnormal processing of APP by γ-secretase results in the accumulation of the β-amyloid peptides which have been identified in AD. Thus, it is generally believed that the reduction of β-amyloid peptides is the key to preventing and/or controlling the symptoms of AD. The instant inventors have accomplished this reduction by de-stabilizing components (presenilin and PEN-2) of the γ-secretase complex and preventing the formation of a functional complex, thus inhibiting the γ-secretase activity and concomitantly reducing the accumulation of β-amyloid peptides in cells of the central nervous system. The method of the instant invention provides a novel approach for prevention and/or treatment of AD and provides an avenue for the development of new compounds for the prevention and/or treatment of AD.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method for modifying Fkbp13 protein interaction with presenilin protein in a cell comprising the steps of:
(a)providing a composition including at least one Fkbp13 protein inhibitor; and
(b) administering the composition of step (a) in an amount effective to achieve modification of said Fkbp13 protein interaction with said presenilin protein in said cell wherein said modification results in a decrease in γ-secretase activity in said cell.

2. The method as in claim 1 wherein said at least one Fkbp13 protein inhibitor is FK506 or rapamycin.

3. The method as in claim 1 wherein said modification de-stabilizes said presenilin protein and prevents formation of a functional γ-secretase complex.

4. The method as in claim 2 wherein said modification de-stabilizes said presenilin protein and prevents formation of a functional γ-secretase complex.

5. A pharmaceutical composition for modifying Fkbp13 protein interaction with presenilin protein in a cell comprising at least one Fkbp13 protein inhibitor combined with a pharmaceutically acceptable solution or carrier.

6. The pharmaceutical composition as in claim 5 wherein said at least one Fkbp13 protein inhibitor is FK506 or rapamycin.

7. A method for reducing D-Amyloid peptide production in a cell comprising the steps of:
(a) providing a composition including at least one Fkbp13 protein inhibitor wherein said at least one Fkbp13 protein inhibitor modifies an interaction between Fkbp13 protein and presenilin protein necessary for formation of a functional γ-secretase complex; and
(b) administering the composition of step (a) in an amount effective to achieve modification of said interaction between said Fkbp13 protein and said presenilin protein, wherein said modification results in a decrease in γ-secretase activity and a reduction in production of β-amyloid peptides in said cell.

8. The method as in claim 7 wherein said at least one Fkbp13 protein inhibitor is FK506 or rapamycin.

9. The method as in claim 7 wherein said modification de-stabilizes said presenilin protein and prevents formation of said functional γ-secretase complex.

10. The method as in claim 8 wherein said modification de-stabilizes said presenilin protein and prevents formation of said functional γ-secretase complex.

11. A method for identifying an agent that inhibits an interaction between Fkbp13 protein and presenilin protein in a cell comprising the steps of:
(a) administering an agent to be assessed for its ability to inhibit an interaction between Fkbp13 protein and presenilin protein to a cell;
(b) determining the extent to which inhibition of an interaction between Fkbp13 protein and presenilin protein occurs in the cell to which the agent of step (a) is administered; and
(c) comparing the extent of inhibition determined in step (b) to the extent of inhibition of an interaction between Fkbp13 protein and presenilin protein in an appropriate control cell, wherein when inhibition of said interaction occurs to a greater extent in the cell to which the agent is administered than in said control cell, the agent is characterized as being effective for inhibiting an interaction between Fkbp13 protein and presenilin protein in said cell.

12. An agent identified by the method of claim 11.
